(19) United States
(12) Patent Application Publication
(10) Pub. No.: US 2021/0236186 A1
(43) Pub. Date: Aug. 5, 2021

(54) PLASMA, MEDIA, SPECIES, SYSTEMS, METHODS

(71) Applicant: Erbe Elektromedizin GmbH, Tuebingen (DE)

(72) Inventors: Matthias Zenker, Tuebingen (DE); Kristin Brunecker, Rottenburg (DE); Alexander Neugebauer, Moessingen (DE); Martin Weiss, Rottenburg-Seebronn (DE)

(73) Assignee: Erbe Elektromedizin GmbH, Tuebingen (DE)

(21) Appl. No.: 17/154,189

(22) Filed: Jan. 21, 2021

(30) Foreign Application Priority Data
Jan. 30, 2020 (EP) 20154694.2

(51) Int. Cl. A61B 18/04

(52) U.S. Cl. CPC A61B 18/042; A61B 2018/00583

(57) ABSTRACT

A plasma for prevention of intraepithelial neoplasia, particularly by anti-viral therapy. The plasma may be used, for example, against the human papillomavirus, for treatment of intraepithelial neoplasia of all grades of severity, e.g., carcinoma in situ lesions, and/or for treatment of invasive carcinoma that is reachable in epithelial manner. A system for creating the plasma includes a medical instrument with at least one electrode having galvanic contact to the plasma. The medical instrument also has an RF device that provides an alternating voltage to supply the instrument with electric power and a gas supply device that is adjusted to supply gas, for example argon gas, to the instrument.

### CROSS REFERENCE TO RELATED APPLICATION

This application claims priority to European Patent Application No. 20154694.2, filed Jan. 30, 2020, the entirety of which is incorporated by reference herein.

### TECHNICAL FIELD

Embodiments of the invention refer to physical plasma, media, species, systems and methods for treatment of illnesses.

### BACKGROUND

Numerous methods for treatment of diseases by means of physical plasma are summarized by the term plasma medicine.

The articles “Cold atmospheric plasma, a novel promising anti-cancer treatment modality” from YAN et al., Oncotarget, 2017, Vol. 8, (No. 9), pp: 15977-15995 as well as “Use of cold atmospheric plasma in oncology: a concise systematic review, Dubuc et al., Ther Adv Med Oncol, 2018, Vol. 10: 1-12 describe research for application of cold plasma under atmospheric pressure (cold atmospheric plasma, CAP) for cancer treatment.

The article “Virucide properties of cold atmospheric plasma for future clinical applications”, Weiss et al., Journal of Medical Virology 89: 952-959 (2017) describes the deactivation of viruses by application of cold plasma under atmospheric pressure.

The article “Physical plasma: a new treatment option in gynecological oncology”, Weiss et al., Archives of gynecology and obstetrics, September 2018 outlines the use of cold physical plasma under atmospheric pressure for the treatment and prevention of gynecological cancer types.

The article “Cold atmospheric plasma (CAP) for anti-cancer applications: Epigenetic effects on DNA integrity and functionality of cervical cancer cells”, describes the facts of cold plasma under atmospheric pressure on cancer cells of the cervix.

The article “Modified argon-plasma coagulation mode and first university center clinical experiences in gastroenterological endoscopy”, Frank et al., Endo heute 2006; 19: 15-22 describes the application of argon-plasma coagulation mode during the treatment of tumors and lesions.

The article “Clinical investigation of the safety and efficacy of a cervical intraepithelial neoplasia treatment using a hyperthermia device that uses heat induced by alternating magnetic fields” Koizumi et al., Molecular And Clinical Oncology, 5: 310-316, 2016 describes the treatment of patients with cervical intraepithelial neoplasia of grade III (CIN III) by means of needles that are heated for the treatment.

WO 02/11634 A1 describes a radio frequency generator for the radio frequency surgery with adjustable power limitation. The generator allows the adjustment of the pulse duration of the modulation signal and/or the pause duration between the modulation signals, such that the peak value of the radio frequency output voltage or the intensity of the electrical light arcs is kept constant.

The poster contribution “Cold atmospheric plasma (CAP) for anti-cancer applications: Epigenetic effects on DNA integrity and functionality of cervical cancer cells”, Weiss et al., for the 62. DGGG-Kongress, Berlin, Oct. 31-Nov. 3, 2018, illustrates the cell growth limiting effect of a CAP-application on cervical SiHa-cells.

In the article “Molecular Effects and Tissue Penetration Depth of Physical Plasma in Human Mucosa Analyzed by Contact- and Marker-Independent Raman Microspectroscopy”, Wenzel et al., published on Oct. 28, 2019 in ACS Appl. Mater. Interfaces, the non-invasive treatment with CAP as promising therapeutical method in precancerous lesions and neoplastic diseases of the human mucosa, e.g., cervical neoplasia, is mentioned. The article describes the results of a study having the goal to examine the use of Raman microspectroscopy to characterize plasma effects on human tissue, which was examined ex-vivo on cervical tissue samples. As described, in a part of the study the effect of CAP to impede cell reproduction on a cell culture having a cell line of cancer cells of cervix is confirmed. The CAP-treatment of the cervix tissue samples as well as the cell line was carried out with an instrument for argon plasma creation operating according to the jet principle. In the context of the study the instrument was dynamically moved over the samples in order to exclude thermical tissue damage.

The website https://clinicaltrials.gov/ct2/show/NCT03218436?term=argon+plasma&cond=CIN&rank=1 describes a study for treatment of cervical intraepithelial neoplasia with physical plasma of low temperature. A CIN of grade I/II that is histologically verified is mentioned as inclusion criteria for the plasma treatment.

In view of this background, a need exists for improved plasma for treatment of diseases as well as systems for creation of such plasma and methods.

### SUMMARY

For solving this object, embodiments of the invention create a plasma (e.g., the plasma according to claim 1), a medium (e.g., the medium according to claim 8), a species (e.g., the species according to claim 9), systems (e.g., systems according to claim 13 or 15) as well as a method (e.g., the method according to claim 16).

According to a first aspect of embodiments of the invention, plasma is created for prevention of intraepithelial neoplasia, particularly for anti-viral therapy, e.g., by plasma-based inactivation of the human papillomavirus, for treatment of intraepithelial neoplasia of all grades of severity, e.g., carcinoma in situ lesions, and/or for treatment of invasive carcinoma that is reachable in epithelial manner. The plasma is preferably an argon plasma, in other cases for example a helium plasma. The system for creation of the plasma comprises a medical instrument with at least one electrode, an RF device for providing an alternating voltage for supply of the instrument with electrical power and a gas supply device that is adjusted to supply gas to the instrument, particularly argon. Preferably at least one electrode of the system has galvanic contact to the plasma. The system creates the plasma preferably as a current conducting plasma.

A second aspect of embodiments of the invention refers to media activated by means of the plasma. Activated means that therapeutically effective species, particularly radicals, are created in the medium by means of the plasma and/or therapeutically effective species, particularly radicals, are introduced in the medium by means of the plasma. For example, the media can be a liquid, particularly suspension, emulsion, particularly tissue liquid, human or animal tissue, particularly neoplastic tissue or, e.g., pasty or rigid material. The media, particularly materials, can be used for treatment of intraepithelial neoplasia. An activated medium that comprises endogenous tissue, e.g., body fluid, can also be referenced as activated target medium. An activated exogenous medium, e.g., a liquid, particularly a solution, suspension or emulsion, a gel or a paste that is applied on or in the tissue of the patient for the treatment or that is supplied to the patient in another manner can be understood as medicine for preventional treatment of the named diseases. If the plasma according to the first aspect is used for creation of an activated endogenous or exogenous medium, the plasma can be used in this sense indirectly for treatment, particularly of one or more diseases mentioned in the context of the first aspect. The radicals can be, e.g., reactive oxygen species (ROS) and/or reactive nitrogen species (RNS). The totality of all reactive oxygen species and all reactive nitrogen species is called RONS.

A third aspect of embodiments of the invention refers to species created by means of the plasma, particularly species introduced in medium mentioned above and/or species created in medium mentioned above, particularly radicals.

In a fourth aspect of embodiments of the invention a system for creation of a plasma according to the first aspect is provided, preferably a current conducting plasma, preferably an argon plasma. The system comprises a medical instrument having at least one electrode with preferably galvanic contact to the plasma and an RF device for providing an alternating voltage for supply of the instrument with electrical power. In addition, the system comprises a gas supply device that can be adjusted for creation of the plasma in order to supply gas, particularly argon, to the instrument. In embodiments the system can be configured for creation of a plasma and thus for prevention and/or treatment of the mentioned diseases by selection of specific system adjustments, such as for example radio frequency (RF) voltage, radio frequency (RF) power, modulation of a radio frequency, gas flow and the like.

At least one electrode of the system can be in galvanic contact with the plasma in that an electrical conductive, particularly metallic surface of the electrode is in contact with the plasma. A current conducting plasma means a plasma with electrode contact such that electrons from the electrode pass over in the plasma. Different to a barrier discharge through a dielectric layer of an electrode, such that the plasma does not have electrode contact, electrons escape from the electrode in the plasma in preferred embodiments of the inventive system for treatment of the mentioned diseases, e.g., by means of prevention. By means of preferred embodiments of the inventive system the discharge can be approached close to the location to be treated, which leads to a high density of neutral and charged, also short-lived species, e.g., reactive oxygen species and/or reactive nitrogen species—comprising particularly radicals—in the plasma at the tissue location and/or in the tissue location to be treated.

In embodiments a warm plasma may be created with the system that allows the non-thermal application of the plasma by movement over the location to be treated, however, can result in a thermal effect, particularly to damage of the functional tissue structure, particularly due to denaturation of proteins during remaining at one location. For example, the warm plasma can have a temperature, particularly ion temperature of more than 45° C., more than 55° C. or even more than 65° C. Particularly at the location next to which the tip of the instrument is positioned without moving it over the tissue, the temperature on the surface of the tissue location can increase to at least 45° C., at least 55° C. or even at least 65° C. For example, the plasma can have a temperature such that only with an application velocity including or above a limit value, e.g., 10 mm/s, with which it is moved over the tissue, the tissue temperature remains less or equal to a limit temperature, e.g., 40° C., particularly preferably less or equal to 37° C. at the tissue location of the human or animal patient to be treated. In order to be able to operate preferably with a manually achievable and controllable application velocity of preferably at most 50 mm/s or less—such that the tissue temperature at the tissue location, over which the plasma is moved remains less or equal to a limit temperature, e.g., 40° C., particularly preferably less or equal to 37° C. or such that a thermal tissue damage, particularly coagulation does not occur due to the plasma treatment—the plasma has preferably a temperature of less than or equal to a limit temperature, wherein the limit temperature can be, e.g., 150° C. or 120° C.

According to a fifth aspect of embodiments of the invention, a method is provided, wherein the method comprises the use of a plasma, e.g., the plasma according to the first aspect, for prevention of intraepithelial neoplasia, particularly by means of an anti-viral therapy, e.g., against the human papillomavirus, for treatment of intraepithelial neoplasia of all grades of severity, for example carcinoma in situ lesions, or for treatment of invasive carcinoma that is reachable in epithelial manner. Similar to using of plasma according to the first aspect and thus also an embodiment of the invention is the use of the medium (e.g., the creation of the medium), the species or the system according to the aspects two to four for one or more of the mentioned treatments.

Particularly the method comprises the non-thermal use of a warm plasma, e.g., embodiments of the plasma according to the first aspect. For this an instrument, e.g., the instrument of the system according to the fourth aspect as explained above by way of example, and thus the plasma is preferably guided over the tissue to be treated so quickly without getting in contact therewith that the temperatures of the tissue locations swiped in this manner remain below a limit temperature, preferably 37° C. or 40° C., such that thermal damage of the tissue is reliably avoided.

Due to the absence of the tissue distraction during the non-thermal application of the plasma, typical risks and complications of laser treatment (bleeding, infection) and conization (bleeding, infection, CK-shortening, 10-times risk for pregnancy and birth complications, etc.) are avoided. The non-thermal treatment is free of pain or only involves minor pain and can therefore be carried out outpatient, as well as without general sedation and without local anesthesia in the usual surgery situation. In many cases already a treatment with a treatment duration of 10 seconds to 10 minutes can be sufficient. Only one person (medically qualified personnel or medical doctor) is needed for the treatment. Sports, intercourse, taking a bath, swimming, professional activities are again possible directly after the treatment.

According to a sixth aspect of embodiments of the invention, a system is provided for creation of a plasma—preferably a current conducting plasma, preferably an argon plasma—having a medical instrument with at least one electrode and having an RF device for providing an alternating voltage for supply of the instrument with electrical power. The electrode has preferably galvanic contact to the plasma. Preferably the systems comprise a gas supply device that is configured to supply gas to the instrument, particularly argon, for creation of a plasma. The system comprises a device for feedback control of the output power, e.g., the output effective power or the output apparent power or the average value of the output actual power of the RF device according to a desired value. The system is preferably intended for use in one of the following therapies—in embodiments this may have occurred by selection of particular system adjustments, such as, e.g., voltage, power, modulation of an RF frequency, gas flow and the like: For prevention of intraepithelial neoplasia, particularly by antiviral therapy, e.g., against the human papillomavirus, for treatment of intraepithelial neoplasia of all grades of severity, e.g., carcinoma in situ lesions, and/or for treatment of invasive carcinoma that is reachable in epithelial manner.

According to a seventh aspect of embodiments of the invention, a plasma is provided for use for prevention of intraepithelial neoplasia, particularly by antiviral therapy, e.g., against the human papillomavirus, treatment of intraepithelial neoplasia of all grades of severity, e.g., carcinoma in situ lesions, and/or for treatment of invasive carcinoma that is reachable in epithelial manner, wherein the plasma is created by means of the system according to the sixth aspect. An eighth aspect of embodiments of the invention refers to a medium activated by means of a plasma according to the seventh aspect for the mentioned therapeutical treatments. A ninth aspect of embodiments of the invention refers to species created by means of the plasma according to the seventh aspect. A tenth aspect refers to a method for prevention of intraepithelial neoplasia, particularly by antiviral therapy, e.g., against the human papillomavirus, treatment of intraepithelial neoplasia of all grades of severity, e.g., carcinoma in situ lesions, and/or for treatment of invasive carcinoma that is reachable in epithelial manner by means of a system according to the sixth aspect, a plasma according to the seventh aspect, a medium according to the eighth aspect and/or with species according to the ninth aspect. Embodiments of the method according to the tenth aspect, particularly refer to the non-thermal use of the plasma according to the seventh aspect. Tantamount in embodiments is the use of the system according to the sixth aspect, the media according to the eighth aspect as well as the species according to the ninth aspect that are part of embodiments of the invention.

Features of the plasma, system, medium, species or the method according to the first to fifth aspects of embodiments of the invention indicated here, can be optional features of the plasma, system, medium, species or method according to the sixth to tenth aspects of embodiments of the invention and vice versa, to provide their advantages. Embodiments of the system according to the sixth aspect, the plasma according to the seventh aspect, the medium according to the eighth aspect and the species according to the ninth aspect correspond to the plasma, system, medium or species according to the first to fourth aspects of embodiments of the invention such that the description with regard to the first to fifth aspect has to be considered for the explanation, particularly of these embodiments.

Additional advantageous embodiments and features of the plasma, media, species, systems and methods according to embodiments of the invention according to the first to tenth aspect are apparent from the following description. Also if plasma, medium, system, method is mentioned in the singular subsequently, always optional features and advantages of all aspects, the first to tenth aspect of embodiments of the invention, are meant as long as the context does not indicate otherwise:

With the plasma according to embodiments of the invention, particularly intraepithelial neoplasia of at least one of the following organ systems of mammals can be treated: Uterine cervix, os, esophagus, stomach, colon, rectum and peritoneum.

The plasma can be particularly used for treatment of intraepithelial neoplasia of the grade I or II of humans, particularly cervical intraepithelial neoplasia.

In spite of decreasing incidence in this part of the world due to highest efforts in prevention and timely radical therapy, 500,000 women come down with invasive cervical carcinoma (CC) each year. About 270,000 patients die per year of this illness and a majority of all patients fight their entire lives with sometimes severe physical and physiological consequences of the disease and therapy.

Cervical carcinoma in situ (CIS) and CC develop from precursor lesions that persist over a duration of mostly multiple years. Beside others, the main risk factor for the development of such cervical intraepithelial neoplasia (CIN) is the local infection with high risk variants of the human papillomavirus (HPV).

The primary therapy of CIN consists of laser evaporation (thermal sclerotherapy) or conization (tissue removal). These methods are mostly carried out under general anesthetic and entail relatively high invasiveness as well as huge clinical and financial efforts. The high incidence of CIN of about 4.2 new diseases per thousand individuals per year results in a huge amount of expensive surgery services. In addition, such surgical interventions for treatment of CIN are sometimes associated with severe bleeding, reduced fertility and a multiple times increased risk for pregnancy complications. The balancing act between overtreatment and concern for manifestation of an invasive CC is a health economic problem. CIN will also pose an ongoing serious problem, due to insufficient HPV vaccination coverage and other risk factors in Germany and throughout the world. Effective and minimum invasive methods without narcotization for the treatment, particularly the CIN, but also other mucosa precancerous changes, are urgently required in the clinical daily routine and currently mostly vacant.

Embodiments of the invention provides remedy here in that these plasma, systems, media, species and methods for a particularly gentle, but effective therapy of particularly CIN, is provided. Preferably the plasma, systems, media, species are used for non-thermal treatment of tissue surfaces extending into the tissue. This means that the temperature at the tissue location to be treated remains particularly preferably always below a critical temperature for thermal tissue damage, e.g., less than or equal to 40° C. or less than or equal to 37° C.

The gas flow of the gas (plasma gas), e.g., argon, serves to create a suitable, as far as possible defined, mixed atmosphere between the distal instrument tip and the tissue such that an ignition of plasma is possible. With too low gas flow too little plasma gas is present between electrode and tissue. The minimum gas flow is the gas flow that has to flow through the instrument at least in order for a plasma to ignite. With too high gas flow too much air or other gas or medium of the environment is added due to turbulence. The maximum gas flow is the gas flow that is allowed to flow through the instrument at most in order for a plasma to ignite. A pure plasma gas atmosphere can presumably make the creation of therapeutically effective species difficult. Thus, the desired gas flow of the gas (plasma gas), e.g., argon, is preferably in a range near the minimum gas flow or in a range near the maximum gas flow for creation of the plasma. The selected gas flow that flows through the instrument can be, for example, at least as high as the minimum gas flow, but less than the maximum gas flow. The gas flow of plasma gas is preferably selected in a range including the minimum gas flow up to including a gas flow that is for example three times larger than the minimum gas flow. The minimum gas flow and also the maximum gas flow can depend from multiple parameters and/or adjustments. The minimum gas flow can particularly depend on a desired treatment distance between the instrument tip and the tissue surface. For example, the treatment distance between the instrument tip and the tissue surface for the treatment can be defined (e.g., 7 mm) and based on the defined treatment distance, the gas flow can be selected that shall flow through the instrument. Provided an inner diameter of the gas channel at the distal end of 2.4 mm, the gas flow can be preferably in a range from including 1 l/min to including 3 l/min, particularly preferably from including 1.3 l/min to including 2.5 l/min, e.g., 1.6 l/min±20%, as an example. These indications refer to normal conditions as well as the further indications with regard to gas flows in this application. Normal conditions are present with a temperature of 0° C. and an atmospheric pressure of 1 atm. A smaller gas flow can be advantageous with smaller inner diameters, but apart therefrom equal parameters and adjustments and a larger gas flow can be advantageous having a larger inner diameter, but apart therefrom equal parameters and adjustments. The inner diameter of the gas supply (e.g. gas channel) of the instrument can have an amount at its distal end of, e.g., including 0.5 mm to including 10 mm, preferably from including 0.8 mm to including 3 mm, particularly 2.4 mm.

In one embodiment the gas flow of, e.g., argon or another plasma gas, of preferably less than 3 l/min, particularly preferably less or equal to 2 l/min, can be advantageous and can result on one hand to a reliable ignition of the plasma, also with low voltage, and on the other hand to a mixture of argon and air between the electrode and the tissue, such that a high density of the reactive species, particularly reactive oxygen species and/or reactive nitrogen species, are created by means of the plasma that are carried with the gas flow to the tissue and/or are penetrated into the tissue.

For creation of an effective plasma instruments are preferred in which the electrical current flow direction corresponds with the plasma flow direction toward the tissue.

A particularly effective plasma can be created by means of a system, if it comprises a neutral electrode, wherein the neutral electrode is arranged on the body of a patient, in order to close the current circuit from the RF device via the electrode, through the plasma and the body of the patient. The plasma is ignited between the electrode on the distal end of the instrument and the tissue of the patient. If the system for creation of an activated medium by means of the plasma shall be used outside of or separate from the body of the patient, the medium can be arranged on a carrier or within a container that serves as neutral electrode. For example, a neutral electrode unit of the system can be electrically conductively connected with an electrically conductive carrier or container for the medium.

Particularly gentle treatment is possible, if the RF device is adjusted to output a limited electrical output effective power of at most 3.5 Watt, particularly preferably at most 2.5 Watt, most particularly preferably at most 2 Watt and yet further preferably at most 1 Watt or less. With a limitation of the output effective power to a maximum value the actual power may have the maximum value, e.g., 2 Watt, or a lower value, e.g., 1 Watt.

In preferred embodiments the RF device is adjusted to supply the electrodes with an alternating voltage that has a radio frequency (RF) of at least 100 kHz, preferably between including 200 kHz up to including 16 MHz, particularly preferably between 300 kHz and 500 kHz, e.g., 350 kHz. The RF frequency can also be referred to as carrier frequency.

The alternating voltage can be pulsed with a fixed or variable mid-frequency (modulation with a mid-frequency), wherein the mid-frequency has an amount of, for example, between including 5 kHz and including 100 kHz, particularly preferably between including 10 kHz and including 50 kHz, e.g., 20 kHz. The pulse duration of each mid-frequency pulse has an amount of preferably one or more RF periods. By means of the pulsation with the mid-frequency, the power of the RF device can be lowered compared with the continuous wave without being required to decrease the peak voltage below a critical value for the ignition.

The alternating voltage is in addition preferably pulsed with a fixed or variable low frequency (modulation with low frequency) that has an amount of, for example, between including 0.5 Hz and including 200 Hz, preferably between including 20 Hz and including 150 Hz, particularly 100 Hz. The pulse duration of each low frequency pulse has an amount of preferably at least one mid-frequency period, preferably between including one and including 50 mid-frequency periods, e.g., 20 mid-frequency periods. If the pulse duration of a low frequency pulse comprises more than one mid-frequency pulse, it can also be called a low frequency pulse packet. The pulsation with low frequency results in addition or as an alternative to the mid-frequency pulsation in a reduction of the output effective power compared with a continuous wave operation of the RF device. The RF device can allow continuous wave operation or not. In addition, the pulsation with low frequency, however, results in an extinction of the plasma in the pulse pauses between the low frequency pulses and consequently in a release of the plasma from the tissue surface. The plasma can thus be moved over the tissue surface without sticking to the tissue surface. In doing so, achievement of a particularly uniform treatment result is facilitated. Thereby also the avoidance of hot tissue locations, i.e. tissue locations with a treatment related temperature above a critical temperature, e.g., of 37° C. or 40° C., due to “sticking” of the plasma at a location with concurrently intensive treatment of tissue locations is facilitated.

The system can be configured to increase the peak output voltage of the RF device at the beginning of a low frequency pulse that can be particularly a package of multiple pulses of the mid-frequency, as long as either an upper limit is reached or a plasma is ignited. The upper limit has an amount of, e.g., between including 2 kV and 6 kV, preferably between 4 kV and 5 kV, e.g., 4.7 kV. The system is preferably further configured that if the ignition has been determined, the voltage of the power supply of the RF device is lowered. In doing so, a reduction of the peak output voltage of the RF device is achieved such that at the beginning of the following low frequency pulse the peak voltage is less than at the point of time of the plasma ignition in the preceding low frequency pulse packet. In doing so, the creation of a too intensive plasma can be avoided that could result in a too strong thermal effect, particularly to thermal tissue damage.

The system preferably comprises a device for feedback control of the output power, e.g., the output effective power or the output apparent power, of the RF device to a desired value, wherein in certain embodiments the plasma is preferably created with feedback control of the output power being activated.

The device for feedback control of the output power can be configured, for example, to adapt the voltage amplitude such that a desired value of an output power of the RF device is reached.

During a modulation with a mid-frequency and a low frequency a pulse packet of the low frequency comprises preferably one or more pulses of the mid-frequency, the pulse packet being followed by a pulse pause. A pulse packet of the mid-frequency comprises preferably one or more oscillation periods of the high frequency followed by a pause. Particularly with small and medium output powers, the pulse packets of the low frequency are so short that a feedback control of the power is impossible by means of known control strategies (voltage feedback control). Without a counter measure the power introduced in the tissue then depends on the voltage, at which the plasma ignites. This ignition voltage depends on external conditions, e.g., distance to the tissue, condition of the electrode or used plasma gas (e.g., helium or argon). The effect of this relation does not result in a clinically relevant difference in the tissue effect for many applications. However, a tissue effect dependent on the distance would be of particular advantage, particularly during the treatment of CIN-lesions. Thus, preferably, as an alternative or in addition to the adaption of the peak voltage, the pulsation (modulation) of the output voltage is adapted. The output voltage can be influenced, for example, by variation of the modulation of the mid-frequency and/or the low frequency and/or by variation of the duration of the low frequency pulse packet in order to feedback control the output power.

The device can thus be configured to feedback control the output power during the creation of the plasma by adjustment of either the peak voltage or else by adjustment of the modulation or else by both measures.

The modulation variation provided by embodiments of the invention preferably instead of the adaption of the peak voltage for adapting the output power can affect the low frequency and/or the mid-frequency. The output power can be detected continuously or in uniform or non-uniform intervals. It is detected preferably after the end of a pulse packet of the low frequency and compared with the desired value. Dependent from the deviation from the desired value, the pulse duration and/or period of the mid-frequency and/or the low frequency can be modified such that the output power approaches the desired value.

The desired value of the average output effective power can have an amount of, for example, at most 3.5 Watt, particularly preferably at most 2.5 Watt, more particularly preferably at most 2 Watt, yet further preferably 1 Watt or less. If the desired value of the average output effective power has an amount of, for example, at most 2 Watt the actual selected desired value can have an amount of, for example, 2 Watt or less, e.g., 1 Watt. The power output can be related, for example, to the average over one period of the low frequency. Alternatively, the power output can be related to an average over the pulse duration of the low frequency, for example. Or the power output can be related to an average over at least one or more mid-frequency pulses or periods, for example. The desired value is preferably less than the maximum RF-output effective power that can be output from the RF device.

Additional advantages and features of embodiments of the inventive plasma, embodiments of the inventive systems, media, species and methods are apparent from the following description as well as the figures. Also if plasma, medium, system, method is mentioned in the singular in the following, always optional features and advantages of all aspects, the first aspect to the tenth aspect, of embodiments of the invention are meant as well, as long as the context does not indicate otherwise. The drawings show:

### BRIEF DESCRIPTION OF THE FIGURES

FIG. 1a—a schematic view of a system with a monopolar instrument and a neutral electrode,

FIG. 1b—an alternative system with an instrument for creation of at least one embodiment an inventive plasma according to the jet principle,

FIG. 2—spin densities achieved by the treatment by means of a system with monopolar instrument and with neutral electrode or by means of a system having an instrument operating according to the jet principle,

FIGS. 3 and 4—schematic illustrations of the use of a plasma that has been created by means of embodiments of an inventive system,

FIG. 5—a tissue surface temperature of a hydrogel sample during an application of atmospheric argon plasma with different application velocities,

FIG. 6—average absolute cell numbers after 24, 72 and 120 hours during non-thermal application of atmospheric argon plasma on cervical carcinoma cell lines in-vitro with different dosages from 0 seconds to 120 seconds,

FIG. 7—a schematic illustration for illustrating RF generators of embodiments of inventive systems, e.g., for a system according to FIG. 1a or FIG. 1b, preferably with a device for feedback control of the output power of the RF generator, and

FIGS. 8a and 8b-schematic illustrations of a pulse sequence of the RF-output voltage of the RF generator according to FIG. 7.

### DETAILED DESCRIPTION

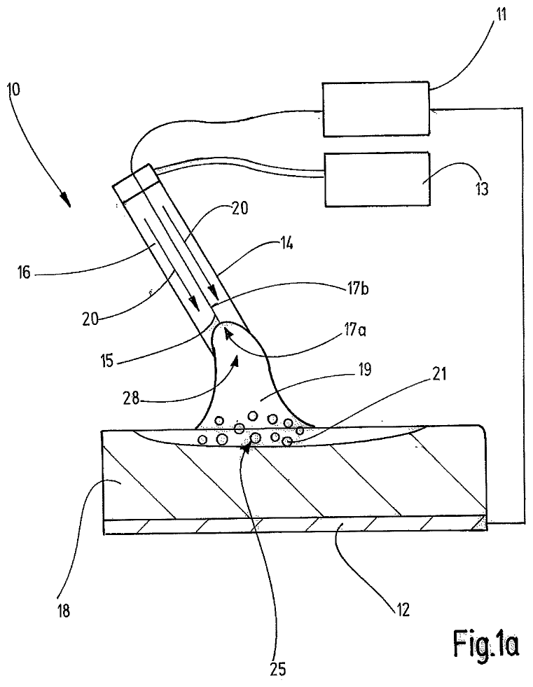

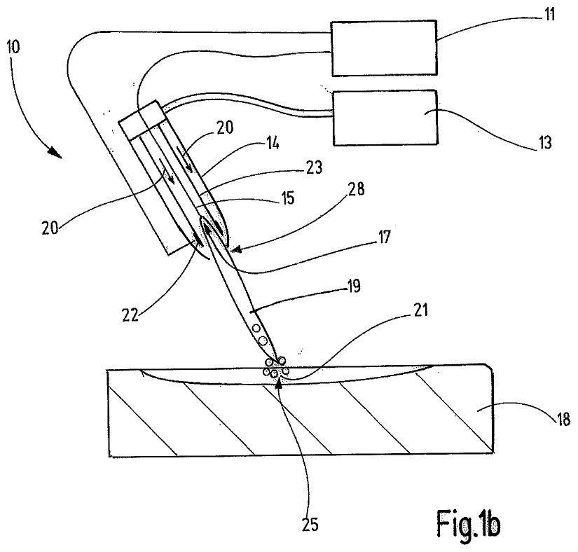

FIG. 1a shows an example of an embodiment of an inventive system 10. The system 10 comprises an RF device 11, a neutral electrode 12, a gas supply device 13 and a medical instrument 14 having an electrode 15, particularly a metal electrode. The electrode 15 can consist, for example, from stainless steel, tungsten or another electrically conductive material. Embodiments are possible in which the electrode 15 comprises at least a metallic surface on a non-metallic core. The electrode 15 can have the shape of a wire, a platelet or a spatula. The distal end 17a of the electrode 15 can be rounded, cylindrically shaped or provided with a tip, as an example. The electrode can be particularly a stainless steel platelet with a tip at the distal end. The electrode 15 of the instrument 14 is preferably free of dielectrical coating. In any case however, the distal end 17a of the electrode 15 is free of dielectrical coating. In addition, the distal end 17a of the electrode 15 as well as an electrode shank 17b adjoining thereto, at least a section of the electrode shank 17b that adjoins the distal end 17a of the electrode 15, is preferably free of dielectrical coating. Preferably not only the distal end 17a of the electrode 15, but also a section of the electrode shank 17b adjoining thereto is free of dielectrical coating. The system 10 does not operate according to the principle of the dielectrical barrier discharge (DBD). The RF device 11 is connected with the instrument 14 in order to supply electric power to the electrode 15. The instrument 14 comprises a gas channel 16 in which the electrode 15 extends longitudinally. The distal end 17a of the electrode 15 can be arranged within the gas channel 16 (as shown), the electrode 15 can end with the gas channel 16 or the electrode 15 can project out of the gas channel 16. The inner diameter of the distal end of the gas channel 16 is preferably between including 0.5 mm and including 10 mm, particularly preferably between 0.8 mm and 3 mm, e.g., 2.4 mm. The gas supply device 13 is fluidically connected with the gas channel 16 in order to apply the gas channel 16 with gas, preferably inert gas, particularly preferably noble gas, e.g., argon. A two-dimensional neutral electrode 12 is part of the system 10 that is electrically conductively connected with a body 18 of the patient over a large area, which is here illustrated highly schematically. The instrument 14 can be an instrument 14 that can be used for the argon plasma coagulation as well.

By application of the instrument 14 with RF voltage by means of the RF device 11, a physical plasma 19 is ignited between the electrode 15 and the body 18 of the patient. The surface of the electrode 15 has galvanic contact to the plasma 19. The electric circuit is closed by the electrode 15, through the plasma 19, through the body 18 of the patient to the neutral electrode 12 and back to the RF device 11. Thereby electrons from the electrode 15 in the instrument 14 enter the current conducting plasma 19 or vice versa. The plasma 19 has galvanic contact to the body 18 of the patient. The electrically conductive body 18 of the patient can thus be considered as second electrode of the system 10 that has galvanic contact to the plasma 19 at the tissue location 21 that is to be treated. In the system 10 according to FIG. 1a, two electrodes 15, 21 have galvanic plasma contact during use. The distance from the first electrode 15 to the tissue 21 is variable without additional measures and depends on the guidance of the instrument 14 by the user. The plasma 19 serves as “conductor piece” for the electrical current from the electrode 15 to the body 18 of the patient. The body 18 of the patient forms another conductor piece for the electrical current between the plasma 19 and the neutral electrode 12. The gas stream 20 supplied by the gas supply device 13 that is at least partly transitioned into the plasma state, displaces at least partly the air or another gas (e.g., pure nitrogen) or gas mixture, forming the atmosphere between the electrode 15 and the tissue location. The system 10 according to FIG. 1a is an example of a system 10 in which the electrical current flow direction corresponds to the plasma flow direction toward the tissue 21.

An example of another embodiment of an inventive system 10 is illustrated in FIG. 1b. Also this embodiment of the system 10 does not operate according to the principle of the dielectric discharge. The system 10 operates according to the jet principle. The system 10 comprises an RF device 11, a gas supply device 13, a medical instrument 14 having a first electrode 15 and a ring-shaped second electrode 22. The RF device 11 is connected with the instrument 14 in order to supply a first electrical voltage to the first electrode 15. The instrument 14 comprises a capillary 23 of an electrically insulating material in which the first electrode 15 extends longitudinally. The second electrode 22 surrounds the end of the capillary 23. The electrode tip 17a of the first electrode 15 is arranged within the capillary 23. The first electrode 15 and the second electrode 22 are arranged in constant distance toward one another. The first electrode 15 and the second electrode 22 are not directly opposed to each other, but the insulating material of the capillary 23 is arranged between the first electrode 15 and the second electrode 22. The gas supply device 13 is fluidically connected with the capillary 23 in order to supply the capillary 23 with gas, preferably inert gas, particularly preferably noble gas, e.g., argon. A neutral electrode 12 is not present in the embodiment of the system 10 according to FIG. 1b. With the plasma 19 being ignited, the circuit is rather closed via the plasma 19 between the first electrode 15 and the second electrode 22. Due to the galvanic contact between the first electrode 15 and the plasma 19, electrons can enter from the first electrode 15 into the plasma 19 or vice versa. The second electrode 22 does not have galvanic contact to the plasma 19 due to the electrically insulating capillary 23 between the first electrode 15 and the second electrode 22. The plasma 19 is blown out from the capillary 23 toward the tissue location 21 to be treated due to the continuously supplied gas flow 20. In addition, the gas flow or plasma flow displaces at least partly the air or another gas (e.g., pure nitrogen) or gas mixture forming the atmosphere at the location between the electrode and the tissue location.

By means of the plasma 19 created with the system 10 according to the first embodiment (FIG. 1a) and by means of the plasma 19 created by the system 10 according to the second embodiment (FIG. 1b), therapeutically effective species 25 (atoms, molecules, ions), particularly neutral or charged radicals are created in the tissue location 21 and/or introduced into the tissue location 21. Particularly the plasma 19 created with the system 10 according to the first embodiment (FIG. 1a) results in a particularly high density of neutral and charged species 25, e.g., reactive oxygen species and/or reactive nitrogen species in the plasma 19 and/or the tissue location 21 to be treated. The tissue location 21 activated in this way forms a medium activated by means of the plasma, wherein the activation results in a regression of neoplasia, for example.

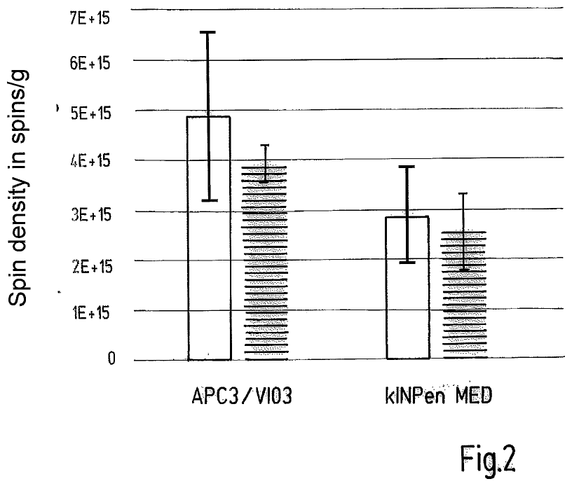

The embodiment of instruments 14 described in connection with FIGS. 1 and 2 can have a leaner configuration compared with the instruments 14 that work according to the principle of dielectric barrier discharge. This particularly applies for embodiments as described in connection with FIG. 1a, because there only one electrode 15 has to be provided on the instrument 14. Also instruments 14, as described in connection with FIGS. 1 and 2, can be moved in greater distance of the distal instrument end 28 over the tissue 21, which facilitates the handling. Embodiments as explained in connection with FIG. 1a distinguish over embodiments, as explained in connection with FIG. 1b in that the at least one plasma channel 19 between the first electrode 15 and the tissue location 21 of the body connected with the neutral electrode 12, radicals are continuously newly created next to the tissue. This also allows particularly short-term radical species to reach the tissue 21. In instruments 14, as explained in connection with FIG. 1b, the plasma creation however occurs further away from the tissue location 21 and the plasma 19 has to be blown out first from the instrument 14. Compared with systems 10 according to embodiments as described in connection with FIG. 1b, a higher number of radicals formed after the same application duration of the plasma 19 on the human or animal tissue—particularly epithelium, or other endogenous or exogenous medium—is considered as advantage of plasma 19 created by means of a system 10 according to embodiments as explained in connection with FIG. 1a, during the treatment of diseases described herein or the creation of activated media for treatment of diseases described herein. The increased number of formed radicals after the same application duration of plasma 19 and the same distance between the respective distal instrument end 28 and the surface 21 to be treated can be proved, for example, by determination of the spin density in the treated samples of the same amount by means of electron spin resonance (ESR) measurement, with which a measure for the number of radicals in the samples can be obtained. Samples of human or animal tissue or solutions with material known as spin traps are eligible as samples, for example. A solution can be formed, e.g., from 0.1 mole DMPO (5,5-Dimethyl-1-pyrrolidin-N-oxide), an example of a spin trap, in degassed DPBS⁺ (composition see below). The tissue samples and/or the solution can be arranged during the treatment with the monopolar instrument 14 of an embodiment of the system 10 according to FIG. 1 on the neutral electrode 12, for example, or the neutral electrode 12 is electrically conductively connected with the sample container. FIG. 2 shows spin densities determined after the treatment of preputial tissue samples with 10 seconds (solid blocks) and 30 seconds (blocks consisting of horizontal lines arranged on top of each other) treatment duration in the tissue samples by using of device parameters “preciseAPC” (pulsed mode with repetition rate of 10 ms, corresponding to 100 Hz low frequency and a mid-frequency of 20 kHz), effect stage 1, 1.6 l/min argon. Effect stage 1 means that the average effective power with which a reference resistance of 1000 Ohm is applied, if it is connected with a potential of the instrument electrode 15 and the potential of the neutral electrode 12, has an amount of at most 2 Watt. With longer treatment duration (compare 30 s treatment duration with 10 s treatment duration) the achieved spin density decreases again. The error bars show a standard deviation. The used system APC3/VIO3 is an example of an embodiment of the system 10 as described in connection with FIG. 1a. The used system kINPen MED is an example of an embodiment of the system 10 as explained in connection with FIG. 1b. The function scheme of the plasma creation in this device is a quartz capillary having an inner diameter of 1.6 mm in the inside of the hand instrument in which a pin-shaped radio frequency electrode with an outer diameter of 1 mm is introduced centrally and to which a radio frequency voltage (1.1 MHz, 2-6 kVpp) is continuously applied ionizing the passing gas with a gas flow of 4.1 l/min used for the comparative experiment. The distance toward the test media was always 7 mm. As apparent from FIG. 2, a higher spin density in the tissue sample can be recognized after a treatment with the system APC3/VIO3 compared with the treatment of a similar tissue probe with the system kINPen MED. The effectivity of the plasma 19 during the treatment of one or more diseases described herein is traced back to the radical species created in the tissue by means of the plasma or inserted into the tissue.

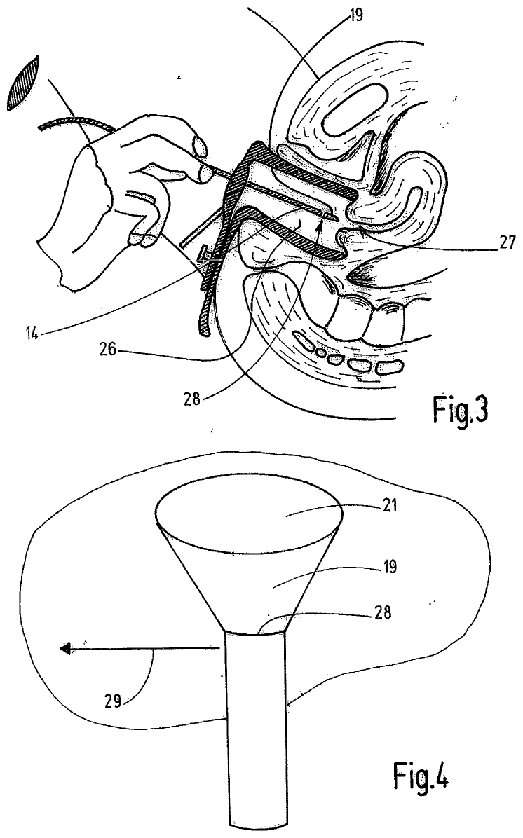

FIG. 3 illustrates the use of embodiments of the inventive plasma 19 for prevention of cervical intraepithelial neoplasia, particularly by anti-viral therapy, e.g., against the human papillomavirus, for treatment of cervical intraepithelial neoplasia of all grades of severity, e.g., cervical carcinoma in situ lesions, and/or for treatment of cervical invasive carcinoma of human beings by means of plasma 19 created by means of the system 10 according to the first embodiment or the system 10 according to the second embodiment (FIGS. 1 and 2). The neutral electrode 12 (not illustrated in FIG. 3) has to be in large area contact with the body 18 of the patient at a suitable location during the treatment by means of the system according to FIG. 1a. The instrument 14 is introduced through the vagina 26 up to or in the uterine cervix 27 and the plasma 19 is ignited. As illustrated in FIG. 4, the plasma 19 swipes over the tissue location 21 to be treated, e.g., due to manual movement (illustrated by arrow 29) of the instrument tip 28 (distal end of the instrument), preferably with a minimum application velocity of, for example, 10 mm/s. The application velocity is the velocity component of the instrument tip 28 parallel to the surface of the tissue location 21. Despite the creation of a warm plasma, due to the movement preferably no thermal damage of the surface of the tissue location 21, particularly no coagulation, results therefrom. The preferred use of the plasma 19 is thus called a non-thermal application. In order to be able to work with a manually achievable and controllable application velocity of preferably at most 50 mm/s or less, such that the tissue temperature at the tissue location 21 over which a plasma 19 is moved remains less or equal to a limit temperature, e.g., 40° C., particularly preferably less or equal to 37° C., such that a thermal tissue damage, particularly coagulation, due to the plasma treatment is avoided, the plasma 19 preferably comprises a temperature of less than or equal to a limit temperature, wherein the limit temperature can be, for example, 150° C. or 120° C.

The gas flow of the gas (plasma gas), e.g., argon, serves to create a suitable, as far as possible defined mixed atmosphere between the distal instrument tip and the tissue, such that an ignition of the plasma 19 is possible. With a too low gas flow, too little plasma gas is present between the electrode 15 and the tissue 21. The minimum gas flow is the gas flow that has to flow at least through the instrument 14 in order for a plasma 19 to ignite. With too high gas flow, too much air or other gas or medium of the environment is added due to turbulence, in order for a plasma 19 to ignite. The maximum gas flow is the gas flow that is allowed to flow through the instrument 14 at most, such that a plasma 19 ignites. Presumably a pure plasma gas atmosphere can make the creation of therapeutic effective species difficult. Thus, the selected gas flow of the gas (plasma gas), e.g., argon, for the creation of the plasma 19 is preferably in a range near the minimum gas flow or in a range near the maximum gas flow. The selected gas flow that flows through the instrument 14 can be, for example, at least as large as the minimum gas flow, but less than the maximum gas flow. The gas flow of plasma gas is preferably selected in a range from including the minimum gas flow to including a gas flow that is, for example, three times larger than the minimum gas flow. The minimum gas flow and also the maximum gas flow can depend on multiple parameters and/or adjustments. The minimum gas flow can particularly depend on a desired treatment distance between the instrument tip and the tissue surface. For the treatment the treatment distance between the instrument tip and the tissue surface (e.g., 7 mm) can be defined, for example, and the gas flow that shall flow through the instrument can be selected based on the defined treatment distance. With an inner diameter of the gas channel at the distal end of 2.4 mm, a treatment distance of, e.g., 7 mm, preferably the gas flow can be in a range from including 1 l/min to including 3 l/min, particularly preferably including 1.3 l/min to including 2.5 l/min, e.g., 1.6 l/min±20%. With smaller inner diameters and apart therefrom identical parameters and adjustments, a smaller gas flow can be advantageous and with larger inner diameters and apart therefrom, identical parameters and adjustments, a larger gas flow can be advantageous.

An embodiment of the invention is illustrated in FIG. 3 based on the treatment of cervical intraepithelial neoplasia of a patient via a transvaginal path. In general, the application for treatment of cervical intraepithelial neoplasia of different organ systems of mammals can be carried out via the transvaginal, oral, parenteral, laparoscopic, intranasal, intrabronchial and rectal path or by means of media or material (medicine) activated by means of embodiments of the inventive plasma 19. If the plasma 19 is used for production of an activated exogenous or endogenous medium 21, the plasma 19 can thus be used indirectly for treatment, particularly of one or more diseases mentioned in this description. During the creation, particularly of activated exogenous medium, the neutral electrode 12 can be formed by a carrier or container (not illustrated) of the medium, if the creation is carried out with a system 10 according to embodiments as they are explained based on FIG. 1a. The non-thermal application of atmospheric argon plasma 19 is suitable for treatment of cervical intraepithelial neoplasia of different pathogenesis and for prophylaxis of invasive carcinoma. Intraepithelial neoplasia comprise here also intraepithelial neoplasia of different organ systems of mammals, e.g., uterine cervix, os, oesophagus, stomach, colon, rectum and peritoneum without being limited thereto. The range of use of embodiments of the inventive plasma 19 comprises also the prophylactic precaution of cervical and other intraepithelial neoplasia, carcinoma in situ (CIS-lesions) and invasive carcinoma of different pathogenesis in mammals predisposed for the respective disease. The treatment comprises the reduction of the disease, stop or delay of progression, induction of a remission of cervical and other intraepithelial neoplasia CIS-lesions and invasive carcinoma of different pathogenesis in mammals predisposed for the respective disease. Combinations with other methods that are useful for the prevention or the treatment of cervical and other intraepithelial neoplasia, CIS-lesions and invasive carcinoma of different pathogenesis are possible.

The proof of the effect of a non-thermal application of atmospheric plasma 19, particularly argon plasma, for treatment of intraepithelial neoplasia is founded on standardized and controlled ex-vivo, in-vitro and in-vivo studies, the procedure of which and the achieved results are described in the following. For the non-thermal application of atmospheric argon plasma 19 in the context of the studies respectively one VIO3/APC3-generator (ERBE Elektromedizin, Tubingen) was used being an example of an RF device 11 and a gas supply device 13. The RF device 11 creates an alternating voltage with a frequency of 350 kHz. As an example of an instrument 14 a FiAPC-probe (ERBE Elektromedizin, Tubingen) with an outer diameter of 3.2 mm and an inner diameter of the distal end of the gas channel 16 of 2.4 mm was used. The probe comprises a stainless steel platelet as electrode having a tip at the distal end. The following parameters have been used: preciseAPC (pulsed mode with a repetition rate of 10 ms, corresponding to 100 Hz low frequency and a mid-frequency of 20 kHz), effect stage 1, 1.6 liters/min argon. Effect stage 1 means that the average effective power has an amount of at most 2 Watt applied to a reference resistance of 1000 Ohm, if it is connected with a potential of the instrument electrode 15 and the potential of the neutral electrode 12.

The examination of the temperature development during application of atmospheric argon plasma 19 was carried out ex-vivo. The examination of the effects on neoplastic cells based on cervical carcinoma cell lines was carried out in-vitro. The examination of the effects on cervical intraepithelial neoplasia of grade I and II was carried out in-vivo.

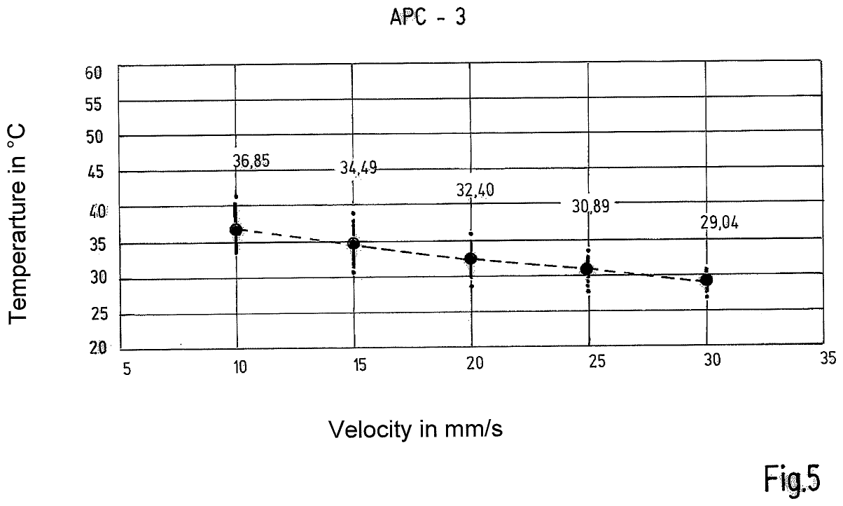

The possibility of non-thermal application of atmospheric argon plasma 19 is based on ex-vivo tissue examinations by means of standardized infrared thermography. Thereby hydrogel samples have been treated with different application velocities with atmospheric argon plasma. Application velocities of more than 10 mm/s thereby showed in the average stable and non-critical tissue temperatures less than 37° C. FIG. 5 shows the tissue surface temperature of hydrogel during an application of atmospheric argon plasma 19 with different application velocities ex-vivo. The results are illustrated as individual measurement points (smaller points) and the average absolute temperature (larger points). A distance of 7 mm between the instrument tip and the surface of the tissue sample has been kept constant. The instrument tip was orientated orthogonal to the surface of the hydrogel samples during the measurements. In order to be able to carry out meaningful thermographic test measurements for the treatment on the surface of human or animal tissue, hydrogel samples have been produced from a 12.5% stock solution of gelatin type B and Dulbecco's Phosphate Buffered Saline (DPBS⁺). DPBS⁺ is a water-based saline solution. It is isotonic such that its ion concentration corresponds to that of the human body. It has a pH-value of 7.0 to 7.3. For production of hydrogel samples, the stock solution was chemically hardened with 1-ethyl-3-(3-dimethylaminopropyl) carbodiimide (EDC) and was placed in a polyactide die (70×20×5 mm) for two hours at 37° C. and 100 rpm in a shaker. After a washing process with pure DPBS⁺ the hydrogel samples were ready to be used for the test measurements.

The results of the standardized infrared thermography show the possibility of a non-thermal application of embodiments of the inventive atmospheric argon plasma 19 with a tissue temperature of <37° C. by using of the described apparatus and adjustments beginning with an application velocity of 10 mm/s.

The effect of a non-thermal application of embodiments of an inventive atmospheric argon plasma 19 on cervical carcinoma cell line (SiHa) was examined in-vitro. The results have been achieved by standardized cell number measurements with a CASY cell counter and analyzer (Roche) after non-thermal application of embodiments of an inventive atmospheric argon plasma 19 with application velocities ≥10 mm/s and different dosages from 0 seconds to 120 seconds and after multiple incubation periods of 24 hours to 120 hours. The plasma treatment was carried out in customary Debulko's modified eagle's medium (DMEM) with 10% fetal calf serum, 1% penicillin/streptomycin. The incubation was carried out under humidified atmosphere (37° C., 5% CO₂, pH 7.4).

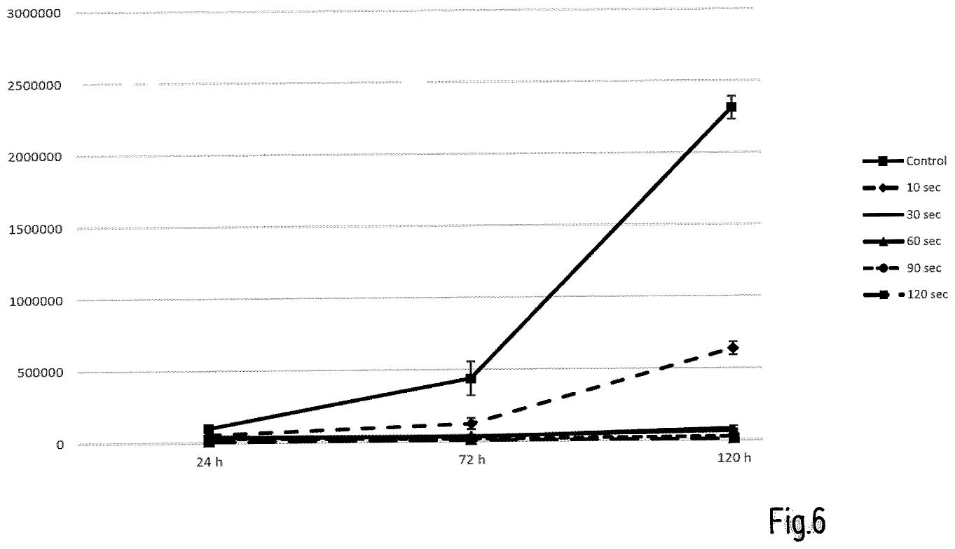

FIG. 6 shows the results of a non-thermal application of atmospheric argon plasma 19 on cervical carcinoma cell lines in-vitro. Illustrated are the average absolute cell numbers from N=6 measurements after 24, 72 and 120 hours carried out independently. The error bars indicate+/−standard deviation.

The in-vitro results of the standardized cell number measurements with a CASY cell counter and analyzer (Roche) after non-thermal application of embodiments of inventive atmospheric argon plasma 19 with application velocities ≥10 mm/s and with different dosages from 0 seconds to 120 seconds show inhibition of proliferation of cervical carcinoma cell lines (SiHa-cells) depending on the dosage and proportion to the dosage within the incubation periods of 24 hours to 120 hours.

The effect of a non-thermal application of atmospheric argon plasma 19 on CIN grade I and II have been examined in-vivo. The ability to effect remission on histologically assured CIN grade I/grade II by non-thermal application of embodiments of inventive atmospheric argon plasma 19 has been analyzed by standardized cytological examinations (Papcytology) after 2 weeks, 3 months and 6 months and histological examinations (external findings by Institute for Pathology, Tubingen) after 3 and 6 months. In the following the procedure is outlined. Patients with histologically assured CIN grade I or grade II have been subject to a clinical colposcopic examination with vinegar-iodine sample in order to present the lesion. Subsequently under colposcopic observation a singular non-thermal application of atmospheric argon plasma 19 has been carried out with application velocities 10 mm/s and depending on the size of the lesion with a minimum dosage of 4 directly subsequent application cycles of 30 seconds in each case. The following parameters have been selected as device adjustment: preciseAPC, effect stage 1, 1.6 liters/min argon. No local and general sedation and analgesia have been used.

Patients >18 years of the Women's Hospital of the University of Tübingen with assured CIN grade I or grade II have been included that have been advised in advance about the finding and possible therapeutic strategies. The patients have been recruited prospectively and not randomized. Applied criteria for the plasma treatment inclusion: age >18 years, histologically assured CIN grade I or grade II, reliably assessable transformation zones of the portio and boundary limitations of the lesions, written agreement for participation in the study after advice. Applied criteria for the plasma treatment exclusion: histologically assured CIN grade III, not completely visible transformation zone, hints for an invasive disease, expected missing compliance of the patient or inability of the patient to understand the sense and purpose of the clinical test, serious cardio-vascular diseases, desire for a classic therapy method, missing patient agreement. As primary terminal point a histologically complete remission of CIN grade I or grade II has been defined. As secondary terminal points a partial histological remission of CIN grade I or grade II, reduced HPV load, pains and life quality, tissue tolerance and compatibility of the plasma treatment have been defined.

The following table shows the histopathological results that have been achieved.

| CIN I/II prior to treatment | Histology 3 months | Histology 6 months |
|---|---|---|
| 43 | 35 of 43 (82%) | 25 of 27 (93%) |

These results show that a non-thermal application of atmospheric argon plasma 19 with application velocities ≥10 mm/s at a dosage of ≥4 directly subsequent application cycles of 30 s in each case comply with a remission rate of CIN I and II lesions of 82% within 3 months. After 6 months 93% of the healed lesions are stable and still unremarkable. For example, in the literature average spontaneous remission rates between 40 and 50% are described for CIN II. In comparison to this the remission rates after non-thermal application of atmospheric argon plasma 19 are significantly higher.

The following table shows the virological results that have been achieved with the system and the adjustments mentioned above.

| High risk HPV positive | |
|---|---|
| prior to treatment | High risk HPV positive 6 months |
| 72% | 11% |

These results show that the non-thermal application of atmospheric argon plasma 19 with application velocities 10 mm/s and a dosage of ≥4 directly subsequent application cycles of 30 s in each case come along with a remission rate of the high risk HPV positivity about 60% within 6 months.

The results of the standardized in-vitro and in-vivo examinations show that the non-thermal application of atmospheric argon plasma 19 is a useful method in treatment of particularly CIN-lesions.

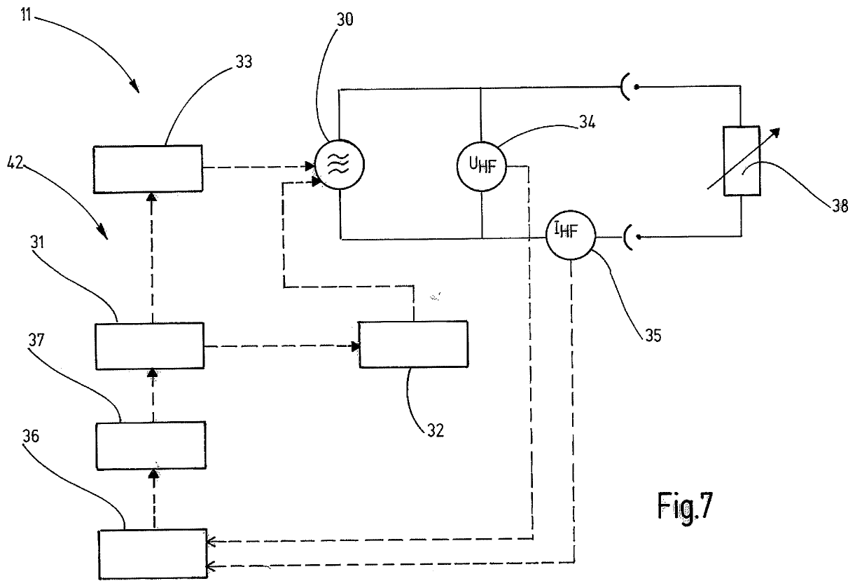

FIG. 7 illustrates an RF device 11 that can be used in embodiments of the inventive system 10, e.g., according to FIGS. 1a and 1b. The RF device 11 comprises an RF generator 30, a control device 31, a modulation device 32 and a power supply 33. The system 10 can comprise a first detection device 34 for determination of a voltage, a second detection device 35 for determination of a current, a signal evaluation device 36 and a comparison device 37. Illustrated is also a load impedance 38 in which, in the case of the system 10 according to FIG. 1a, the impedance of the plasma 19 and the body 18 between the plasma 19 and the neutral electrode 12 and in the case of the system 10 according to FIG. 1b, the impedance of the plasma 19 is included.

The RF generator 30 is configured to generate an RF voltage that can have an RF frequency, e.g., of at least 100 kHz, preferably between including 200 kHz and including 16 MHz, particularly preferably between 300 kHz and 500 kHz, e.g., 350 kHz.

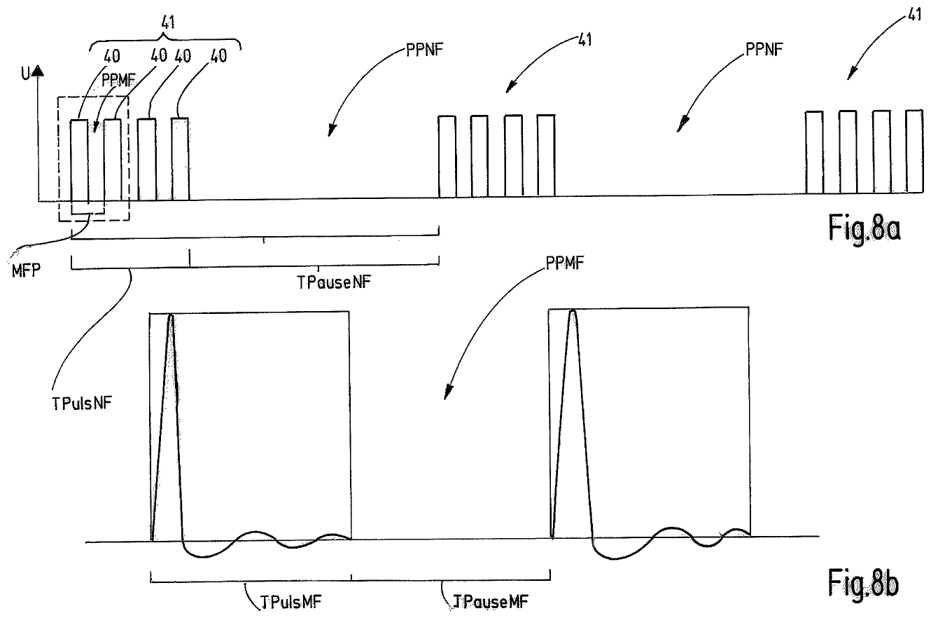

FIG. 8a illustrates schematically and by way of example a modulated radio frequency output alternating voltage U of the RF generator 30 in the progress of time. FIG. 8b shows a section of FIG. 8a.

The alternating voltage U is modulated by means of the modulation device 32, preferably with a mid-frequency (MF) and/or a low frequency (NF). The mid-frequency can have an amount of, e.g., between including 5 kHz and including 100 kHz, particularly preferably between including 10 kHz and including 50 kHz, e.g., 20 kHz. The pulse duration TPulsMF of the mid-frequency pulse 40 has an amount of preferably at least one or more RF periods.

The low frequency has an amount of, for example, between including 0.5 Hz and including 200 Hz, preferably between including 20 Hz and including 150 Hz, particularly 100 Hz. The pulse duration TPulsNF of the low frequency packages 41 has an amount of preferably at least one mid-frequency period MFP, preferably between including one mid-frequency period and 50 mid-frequency periods, e.g., 20 mid-frequency periods.

The modulation with the mid-frequency and/or the low frequency can be used to reduce the output power compared with a non-pulsed alternating voltage, e.g., with predefined minimum peak voltage. The modulation or pulsation with the low frequency results in an extinction of plasma 19 in the pulse pauses PPNF of the low frequency and thus to a release of the plasma 19 from the tissue location 21. In doing so, the plasma 19 does not stick to one tissue location 21 in spite of the movement of the tip 28 of the instrument 14 longitudinally parallel with distance to the tissue location 21. Rather the tissue location 21 to be treated can be uniformly swiped with the plasma 19.

For example, in the mode preciseAPC, effect stage 1 of the RF device APC3/VIO3 (an example for the system according to FIG. 1a) mentioned above that was used for treatment of patients, the mid-frequency MF and the low frequency NF are non-variably predefined (MF=20 kHz and NF=100 Hz), such that during operation with a non-varying reference resistance without plasma path, the predefined average power is obtained for the respective effect stage. The selection of the effect stage (1 to 10) influences the non-varying duration TPulsNF of each low frequency pulse. If the user holds the instrument tip 28 adjacent to the tissue location 21 and activates the plasma generation, the RF device 11 selects independent from the effect stage the output voltage so high that the plasma 19 is ignited. In the mentioned mode preciseAPC, e.g., effect stage 1, the system VIO3, as an example for the system 10, is configured to increase the peak output voltage of the RF device 11 at the beginning of a low frequency pulse that is a packet of multiple pulses of the mid-frequency, as long as either an upper limit is reached or a plasma is ignited. The upper limit has an amount of, for example, between including 2 kV and 6 kV, preferably 4 kV and 5 kV, in systems with VIO3 4.7 kV, for example. The system 10 is further configured such that the output voltage of the power supply 33 of the RF device 11 is lowered, if the system 10 has recognized ignition. Particularly the output voltage of the power supply 33 is feedback controlled on a desired value that is smaller than the value required for the ignition. In doing so, a reduction of the peak output voltage of the RF generator 30 is achieved such that at the beginning of the subsequent low frequency pulse the peak voltage is less than at the point of time of the plasma ignition in the preceding low frequency pulse packet. In doing so, the creation of a too intensive plasma can be avoided with the system VIO3 that could lead to a too strong thermal effect, particularly to thermal tissue damage.

However, the ignition voltage is dependent from the distance to the tissue 21 and apart therefrom, for example, also from characteristics of the electrode 15 (e.g., geometry and/or material), the used gas, characteristics of the tissue and the environment. The distance dependency means that the average output voltage of the RF device 11 depends on the distance of the electrode tip 17a from the tissue 21 in the same effect stage 1. For example, the average output effective power of the RF device 11 can have an amount for a distinct effect stage, e.g., effect stage 1, about 1.5 Watt at 3 mm distance, about 5 Watt at 12 mm distance. This poses particular requirements on the guidance of the instrument tip 28 by the user over the tissue 21 in always the same distance and with constant velocity as far as possible in order to achieve a uniform tissue effect. Because during movement over the tissue 21 it comes to repeated ignition at the beginning of each pulse packet 41 of the low frequency at potentially different distances of the electrode tip 17a to the tissue 21 due to the modulation with the low frequency.

Therefore, according to embodiments of the invention a system 10 is proposed (this is also illustrated by means of FIG. 7) that comprises a device 42 for feedback control of the output power, e.g., the output effective power or the output apparent power of the RF device. The system 10 can have a control for the reduction of the peak output voltage, as illustrated above in connection with the explanation of the system VIO3.

For this the device 42 comprises a unit for determination of the output effective power. For example, the device can be configured to determine the output power of the power supply 33 and to determine the output effective power of the RF generator 30 based on a known efficiency of the RF generator 30. As an alternative or in addition, the device 42 can be configured to determine the output voltage of the RF generator 30 by means of the first detection device 34, as illustrated in FIG. 7 and to determine the output current of the RF generator 30 by means of the second detection device 35. By means of the signal evaluation device 36, the device 42 can be configured to determine the output power of the RF generator 30 from the measurement values of the first detection device 34 and the second detection device 35.

The determination of the output power and the adjustment of the modulation can be carried out continuously or in uniform or non-uniform time periods. The output power can be determined by averaging the product of the voltage and current put in phase relation to each other over one or more periods, e.g., one or more mid-frequency periods and/or low frequency periods. The determination of the output power is then carried out preferably in the following pulse pause, particularly pulse pause PPNF of the low frequency or pulse pause PPMF of the mid-frequency. The determination can be carried out regularly in the pulse pauses PPNF of the low frequency, for example. Particularly, the output power can be averaged over one or more mid-frequency periods until the end of a low frequency pulse packet and the average value of the output power can be determined therefore as actual value of the output power after the end of a low frequency pulse packet.

The device 42 can be configured to compare the determined output power (actual value), e.g., the output effective power with a desired value of the output power, e.g., for the output effective power, by means of the comparison device 37 and can be configured to carry out one or more modifications in case of deviations of the actual value from the desired value by means of the control device 31 in order to approach the actual value to the desired value.

In addition, or as an alternative, it would be basically possible to modify the peak value of the output voltage of the RF generator 30 depending on the determined deviation of the actual value from the desired value for control purposes. This however requires a high control speed in case of very short pulse packages 41 of the low frequency that is only difficult to realize. In addition, a reduced peak voltage can result in a remarkably degraded ignition behavior. Therefore, it is preferably refrained from modifying the peak value of the output voltage for control and the modulation of the RF frequency dependent on the deviation of the actual value from the desired value of the output power, particularly the output effective power, is modified instead. Basically, a control is possible as an alternative in which the peak value of the output voltage is modified such that it is above a minimum value and in addition or as an alternative, the modulation is changed.

In embodiments a detection of the actual value of the output power in a pulse packet of the low frequency and an adaption of the modulation is possible so quickly by the power feedback control by means of adaption of the modulation, such that the desired value of the output power is achieved within the same pulse packet or within or at the beginning of the first subsequent pulse packet of the low frequency. The modification of the modulation can particularly comprise the low frequency modulation and/or the mid-frequency modulation. For example, the pulse-pause-ratio of the low frequency pulse packages 41 can be modified by modification of the pulse duration TPulsNF, modification of the pause duration TPauseNF and/or modification of the low frequency period. As an alternative or in addition, the pulse-pause-ratio of the mid-frequency pulses 40 can be modified by modification of the pulse duration TPulsMF, modification of the pause duration TPauseMF and/or modification of the mid-frequency period.

With the system 10 having the device 42 for feedback control of the output power, e.g., the output effective power, a plasma 19 with constant output power of the RF generator can be created independent from ignition conditions, particularly the distance to the tissue 21, which simplifies the guidance of the instrument 14 for effective non-thermal treatment remarkably, as for example illustrated in FIGS. 3 and 4. A system 10 with a device 42 for feedback control of the output power or a plasma 19 created therewith can be advantageously particularly used for treatment of one or more of the above-mentioned diseases. In general, a system 10 with a power feedback control as described in connection with the device 42 can be advantageously used everywhere, where a distance independent reproducible tissue effect is desired. Due to the power feedback control, the tissue effect can be regulated particularly sensitively. A field of use can be, for example, the non-thermal wound treatment, where a thermal damage of the wounded tissue shall be avoided.

According to embodiments of the invention, particularly a plasma 19 is created for prevention of intraepithelial neoplasia, particularly by anti-viral therapy, e.g., against the human papillomavirus, for treatment of intraepithelial neoplasia of all grades of severity, e.g., carcinoma in situ lesions, and/or for treatment of invasive carcinoma that is reachable in epithelial manner, wherein the plasma is created with the system 10 for creation of a plasma 19, preferably an argon plasma, having a medical instrument 14 with at least one electrode 15 with galvanic contact to the plasma 19, an RF device 11 for providing an alternating voltage for supply of the instrument 14 with electric power and a gas supply device 13 that is adjusted to supply gas, particularly argon, to the instrument 14.

1. A plasma for prevention of intraepithelial neoplasia, particularly by anti-viral therapy, e.g., against the human papillomavirus, for treatment of intraepithelial neoplasia of all grades of severity, e.g., carcinoma in situ lesions, and/or for treatment of invasive carcinoma that is reachable in epithelial manner, wherein the plasma is created with a system for creation of a plasma, preferably an argon plasma, having a medical instrument with at least one electrode with galvanic contact to the plasma, an RF device for providing an alternating voltage for supply of the instrument with electric power and a gas supply device that is adjusted in order to supply gas, particularly argon, to the instrument.

2. The plasma according to claim 1 for treatment of intraepithelial neoplasia of at least one of the following organ systems of mammals: Uterine cervix, os, esophagus, stomach, colon, rectum and peritoneum.

3. The plasma according to claim 1 for treatment of cervical intraepithelial neoplasia of the grade I to III.

4. The plasma according to claim 1, wherein the system comprises a neutral electrode, wherein the neutral electrode is arranged on the body of a patient in order to close the circuit from the RF device via the electrode of the instrument through the plasma and the body of the patient, wherein the plasma between the electrode at the distal end of the instrument and the tissue of the patient is ignited.

5. The plasma according to claim 1, wherein the RF device is adjusted to supply an alternating voltage to the electrode,
the alternating voltage having a radio frequency of at least 100 kHz and being pulsed with a mid-frequency, wherein the mid-frequency has in the inclusive range of 5 kHz to 100 kHz,
wherein the pulse duration of the mid-frequency pulses has an amount of one or more RF periods,
wherein the mid-frequency are pulsed with a frequency in the inclusive range of 0.5 Hz to 200 Hz, and
wherein the pulse duration of the low frequency pulses has an amount of at least one mid-frequency period, preferably between including one and including 50 mid-frequency periods, e.g., 20 mid-frequency periods.

6. The plasma according to claim 1, wherein the system comprises a device for feedback control of the output power of the RF device.

7. The plasma according to claim 1, wherein the gas supply device is configured to supply the instrument with a flow in a range of including a minimum gas flow to including a gas flow that is three times larger than the minimum gas flow.

8. A medium in which species are created and/or introduced by means of a plasma according to claim 1 for at least one selected from the group of prevention of intraepithelial neoplasia, treatment of intraepithelial neoplasia of all grades of severity, and treatment of invasive carcinoma that is reachable in epithelial manner.

9. Species created by means of a plasma according to claim 1 for at least one selected from the group of prevention of intraepithelial neoplasia, treatment of intraepithelial neoplasia of all grades of severity, and treatment of invasive carcinoma that is reachable in epithelial manner.

10. A system for creation of a plasma according to claim 1.

11. A system for creation of a medium according to claim 8.

12. A system for creation of a species according to claim 9.

13. A system for creation of a plasma, preferably a current conducting plasma, preferably an argon plasma, having a medical instrument with an electrode and an RF device for providing an alternating voltage for supply of the instrument with electric power, wherein the system comprises preferably a gas supply device that is adjusted in order to supply gas, particularly argon, to the instrument, wherein the system comprises a device for feedback control of the output power, preferably the output effective power, of the RF device to a desired value.

14. The system according to claim 13, wherein the RF device is adjusted to apply an alternating voltage to the electrode,
having an RF frequency of at least 100 kHz,
wherein the alternating current is pulsed with a mid-frequency, wherein the mid-frequency has an inclusive range of 5 kHz to 100 kHz,
wherein a pulse duration of the mid-frequency pulses has an amount of one or more RF periods,
wherein the alternating current is modulated with a low frequency having an inclusive range of 0.5 Hz to 200 Hz, wherein the pulse duration of the low frequency pulses has at least one mid-frequency period,
wherein the device is configured to feedback control the output power by means of adaption of the modulation of the mid-frequency and/or the low frequency.

15. The system according to claim 13, wherein the desired value of the output effective power of the RF device is at most 3.5 Watt.

16. A method for plasma-based prevention of at least one selected from the group of intraepithelial neoplasia, treatment of intraepithelial neoplasia of all grades of severity, and treatment of invasive carcinoma that is reachable by means of the system according to claim 11, wherein the method comprises swiping the plasma over a tissue location, wherein the plasma has a temperature of more than or equal to 45° C., with an application velocity equal to or above a limit value, such that the temperature of the tissue location remains below or equal to a limit temperature.

17. A method for plasma-based prevention of at least one selected from the group of intraepithelial neoplasia, treatment of intraepithelial neoplasia of all grades of severity, and treatment of invasive carcinoma that is reachable by means of a plasma according to claim 1,
wherein the method comprises swiping the plasma over a tissue location, wherein the plasma has a temperature of more than or equal to 45° C., with an application velocity equal to or above a limit value, such that the temperature of the tissue location remains below or equal to a limit temperature.

18. A method for plasma-based prevention of at least one selected from the group of intraepithelial neoplasia, treatment of intraepithelial neoplasia of all grades of severity, and treatment of invasive carcinoma that is reachable by means of the system according to claim 13, wherein the method comprises swiping the plasma over a tissue location, wherein the plasma has a temperature of more than or equal to 45° C., with an application velocity equal to or above a limit value, such that the temperature of the tissue location remains below or equal to a limit temperature.

19. A plasma for carrying out the method according to claim 16.

20. A plasma for carrying out the method according to claim 18, wherein the plasma is a plasma for prevention of intraepithelial neoplasia, particularly by anti-viral therapy, e.g., against the human papillomavirus, for treatment of intraepithelial neoplasia of all grades of severity, e.g., carcinoma in situ lesions, and/or for treatment of invasive carcinoma that is reachable in epithelial manner, wherein the plasma is created with a system for creation of a plasma, preferably an argon plasma, having a medical instrument with at least one electrode with galvanic contact to the plasma, an RF device for providing an alternating voltage for supply of the instrument with electric power and a gas supply device that is adjusted in order to supply gas, particularly argon, to the instrument.
